(19) United States
(12) Patent Application Publication
(10) Pub. No.: US 2004/0224039 A1
(43) Pub. Date: Nov. 11, 2004

(54) Compositions and methods for the treatment of diseases and disorder associated with oxidative damage

(76) Inventor: Donald Brucker, La Jolla, CA (US)

Correspondence Address: FISH & RICHARDSON, PC, 12390 EL CAMINO REAL, SAN DIEGO, CA, 92130-2081, US

(21) Appl. No.: 10/777,993

(22) Filed: Feb. 11, 2004

Related U.S. Application Data
(60) Provisional application No. 60/447,146, filed on Feb. 12, 2003.

(51) Int. Cl. A61K 35/78; A61K 38/05; A61K 31/7076; A61K 31/60

(52) U.S. Cl. 424/771; 514/18; 514/47; 514/165; 514/570

(57) ABSTRACT

Provided are methods and compositions useful for treating disease and disorder associated with oxidative damage and the like. The methods and compositions are useful in the treatment of such disorders as Parkinson's disease. The compositions comprise an aqueous medium having dispersed or dissolved therein NAD (or a derivative thereof), glutathione (or a derivative thereof) and may further include an herbal therapeutic agent and/or an analgesic agent, wherein the composition is effective when delivered to the mucosal membrane.

### CROSS REFERENCE TO RELATED APPLICATIONS

This application claims priority under 35 U.S.C. §119 to U.S. Provisional Application Serial No. 60/447,146, filed Feb. 12, 2003, the disclosure of which is incorporated herein by reference in its entirety.

### TECHNICAL FIELD

This disclosure relates to compositions and methods for treating disease and disorder associated with oxidative damage, and more particularly to methods and compositions for treating Parkinson's Disease.

### BACKGROUND

Excessive concentrations of various forms of oxygen and of free radicals can cause damage to living systems, including the peroxidation of membrane lipids, the hydroxylation of nucleic acid bases, and the oxidation of sulfhydryl groups and of other sensitive moieties in proteins. If uncontrolled, mutations and cellular death result.

Free radicals, particularly free radicals derived from molecular oxygen, have been associated with a number of diseases and disorders (Zimmermen J. J. (1991) Chest 100: 189S). Some of the disease and disorders associated with oxygen free radicals include pulmonary oxygen toxicity, adult respiratory distress syndrome (ARDS), bronchopulmonary dysplasia, sepsis syndrome, and a variety of ischemia-reperfusion syndromes, including myocardial infarction, stroke, cardiopulmonary bypass, organ transplantation, necrotizing enterocolitis, acute renal tubular necrosis, and other disease.

Many free radical reactions are highly damaging to cellular components; they crosslink proteins, mutagenize DNA, and peroxidize lipids. Once formed, free radicals can interact to produce other free radicals and non-radical oxidants such as singlet oxygen and peroxides. Degradation of some of the products of free radical reactions can also generate potentially damaging chemical species. For example, malondialdehyde is a reaction product of peroxidized lipids that reacts with virtually any amine-containing molecule. Oxygen free radicals also cause oxidative modification of proteins (Stadtman E. R. (1992) Science 257: 1220).

### SUMMARY

The disclosure provides a composition comprising an aqueous medium having dispersed or dissolved therein a glutathione and an NAD or derivative thereof, wherein the glutathione is at an amount from about 0.01% to 0.9%, and wherein the NAD or derivative thereof is at an amount from about 0.01% to 0.9%.

The disclosure also provides a method of treating a subject suffering from an oxidative disease or disorder, comprising administering a composition of the disclosure in an amount sufficient to reduce or ameliorate the oxidative disease or disorder.

The disclosure also provides a method of treating a subject suffering from Parkinson's disease, comprising administering to a mucus membrane of the subject a composition comprising: an aqueous medium; a glutathione and an NAD or derivative thereof, wherein the glutathione is at an amount from about 0.01% to 0.9%, and wherein the NAD or derivative thereof is at an amount from about 0.01% to 0.9% in the aqueous medium; and wherein the composition is administered in an amount effective to reduce the severity and/or treat the symptoms of Parkinson's disease.

The disclosure also provides a nasal spray and a sublingual spray comprising the composition of the disclosure.

Other features, objects, and advantages will be apparent from the description and from the claims.

### DETAILED DESCRIPTION

The disclosure provides compositions and methods for treating diseases and disorders associated with oxidative damage. A disease or disorder associated with oxidative damage includes pathological conditions in a subject that results at least in part from the production of, or exposure to, free radicals, particularly oxygen free radicals, and other reactive oxygen species in vivo. Thus, the methods and compositions of the disclosure are useful in treating pathological states that result from free radicals or reactive oxygen species including diseases and disorders such as, for example, ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, uveitis, emphysema, gastric ulcers, oxygen toxicity, neoplasia, radiation sickness, and other pathological states including neurological diseases and disorders (e.g., Parkinson's Disease (PD)).

Parkinson's disease (PD), known also as striatal dopamine deficiency syndrome, is a degenerative disorder of the central nervous system characterized by muscular rigidity, tremor at rest, akinesia, and postural abnormalities. In early stages of PD, there appears to be a compensatory increase in the number of dopamine receptors to accommodate the initial loss of dopamine neurons. As PD progresses, the number of dopamine receptors decreases, apparently due to the concomitant degeneration of dopamine target sites on striatal neurons. The loss of dopaminergic neurons in PD results in enhanced metabolism of neurotoxic hydroxyl radicals (OH′). The generation of free radicals can also be produced by 6-hydroxydopamine (6-OHDA) or 1 methyl-4-phenyl-1,2,3,6 tetrahydropyridine (MPTP) which destroys striatal dopaminergic neurons causing PD in experimental animals as well as human beings. Studies of the substantia nigra after death in PD have suggested the present of oxidative stress and depletion of reduced glutathione (Sian et al. Ann Neurol. 36(3):348-55, 1994; and Ebadi et al., Neuroendo. Lett. 18:111-122, 1998).

Parkinson's disease is one of a larger group of neurological conditions called motor system disorders. In the normal brain, some nerve cells produce the chemical dopamine, which transmits signals within the brain to produce smooth movement of muscles. In Parkinson's patients, 80 percent or more of these dopamine-producing cells are damaged, dead, or otherwise degenerated. This causes the nerve cells to fire wildly, leaving patients unable to control their movements. Symptoms usually show up in one or more of four ways: tremor or trembling in hands, arms, legs, jaw, and face; rigidity or stiffness of limbs and trunk; bradykinesia or slowness of movement; postural instability or impaired balance and coordination.

The methods and compositions are useful in treating or reducing symptoms associated with oxidative damage. By treating or reducing symptoms associated with a disease or disorder is meant a detectable benefit by decreasing symptoms, increasing survival, or providing other detectable clinical benefits in treating or preventing a pathological state associated with the disease or disorder.

Free radicals are atoms, ions, or molecules that contain an unpaired electron (Pryor, W. A., Free Radicals in Biol. 1: 1, 1976). Free radicals are usually unstable and exhibit short half-lives. Elemental oxygen is highly electronegative and readily accepts single electron transfers from cytochromes and other reduced cellular components; a portion of the O₂ consumed by cells engaged in aerobic respiration is univalently reduced to superoxide radical (O₂⁻) (Cadenas E., Ann. Rev. Biochem. 58: 79, 1989). Sequential univalent reduction of O₂⁻ produces hydrogen peroxide (H₂O₂), hydroxyl radical (.OH), and water.

Provided are methods and compositions useful in treating disorder cause by oxidative damage including Parkinson's disease and the like. The methods and compositions use naturally occurring substances that are delivered by routes resulting in reduced side-effects and improved beneficial uptake. Such methods and compositions are based, in part, upon hormesis and homeopathy.

Hormesis is a hypothesis that states that most, if not all, chemical and physical agents have the capacity to stimulate biological effects at doses below the toxicity threshold, while causing toxicity at doses above the threshold. A number of recently identified agents have been shown to be administered at levels that induce a biological effect, however, when the doses are decreased to a minimal level an opposite therapeutic effect is identified.

In addition, interest in homeopathic and/or herbal medicines has increased recently due in part to the lower cytotoxicity associated with such medications. Homeopathy is commonly used to mean a system of medicine based on the use of infinitesimal doses of medicines capable of producing symptoms similar to those of the disease treated. By stimulating a subject's natural defenses (i.e., increasing the symptoms) the subject will be motivated or directed towards homeostasis, since one's symptoms are actually efforts of the organism to reestablish homeostasis or balance. Homeopathic treatment encompasses some forms of natural materials including plant extracts and the like. However, some agents that are referred to as homeopathic treatments are not necessarily homeopathic because the agents do not stimulate disease or disorder symptoms but rather inhibit their onset or severity. The non-disease stimulating agents are still typically administered in infinitesimally small doses and thus are sometimes considered “homeopathic”.

Traditionally the delivery of medicines and/or therapeutics has been by oral administration through the gastrointestinal tract in the form of tablets or by edible leaves, teas, tinctures, or extracts. It should be noted that the degradative nature of the gastrointestinal tract results in the degradation of the active ingredients in many of such herbal medicines and therapeutic agents. In order to overcome the degradative processes inherent in oral administration, the amounts and concentrations of medicines and/or therapeutics, including active ingredients, are increased such that a desired amount of the active ingredient is available to treat the subject once it traverses the gastrointestinal tract. However, such an increase in concentrations and amounts may cause additional side-effects including gastrointestinal discomfort, oral sores, and the like. Alternatively, the therapeutic agents are administered by intravenous routes. As IV routes are highly invasive, painful, and run the risk of infection, bruising and the like.

The disclosure provides methods and compositions that utilize small amounts of active ingredients utilizing the theories of hormesis and homeopathy. The routes of administration used in the methods and compositions of the disclosure do not require excessive amounts of the medicine or active ingredient. The disclosure provides compositions for administration to a mucus membrane of a subject including, for example, the sinonasal cavity or oral mucosa (e.g., sublingual space). The mucosal tissue does not contain the acids and enzymes present in the gastrointestinal tract and does not require the invasiveness of needle administrations. Thus, the therapeutic agents disclosed herein, as well as the active ingredients thereof, are readily available for absorption into the blood stream and related tissues of the subject without unwanted degradation, pain, infection, and/or bruising. Accordingly, smaller amounts of such medicines and/or active ingredients are useful in order to treat a subject afflicted with a disease or disorder associated with oxidative damage (e.g., Parkinson's Disease). The smaller amounts used in the compositions and methods of the disclosure also result in less gastrointestinal discomfort, sores, and the like. In addition, administration to the mucus membrane results in a faster uptake of the medicinal product and/or active ingredient.

The therapeutic agents useful in the compositions and methods of the disclosure include those derived from, for example, glutathione and glutathione like substances as well as NAD and derivatives thereof (e.g., NADH). NAD and NAD derivatives include quinolinic acid; quinolinic acid ribonucleotide; nicotinamide; nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof. All of these chemicals are commercially available or are generally known. Typically the NAD related molecule is nicotinamide or nicotinic acid, more typically nicotinamide. Pharmaceutically acceptable salts are also included and can be derived from a variety of organic and inorganic counter salts well known in the art and include, by way of example only, sodium, potassium, calcium magnesium, ammonium, tetralkylammonium, and the like.

The ubiquitous tripeptide L-glutathione (GSH) (gamma-glutamyl-cysteinyl-glycine), is a well known agent. When oxidized, it forms a dimer (GSSG), which may be recycled in organs having glutathione reductase. Glutathione may be transported through membranes by the sodium-dependent glutamate pump (Tanuguchi, N., et al. Eds., Glutathione Centennial, Academic Press, New York (1989), incorporated herein by reference).

GSH is known to function directly or indirectly in many important biological phenomena, including the synthesis of proteins and DNA, transport, enzyme activity, and metabolism. GSH is synthesized by most cells, and is also supplied in the diet. Because of the existing mechanisms for controlling interconversion of reduced and oxidized glutathione, an alteration of the level of reduced glutathione (GSH), e.g., by administration of GSH to an organism will tend to shift the cells of the organism to a more reduced redox potential. Likewise, subjecting the organism to oxidative stress or free radicals will tend to shift the cells to a more oxidized potential.

General methods of formulating an aqueous medium for administration of the compositions of the disclosure can be found in, for example, “Remington's Pharmaceutical Sciences.” For example, formulations for inhalation may contain aqueous solutions comprising, polyoxyethylene-9-lauryl ether, glycocholate, and deoxycholate, or may be oily solutions for administration in the form of nasal drops, or as a gel to be applied intranasally.

The aqueous medium typically comprises water and typically includes other materials such as surfactants, vitamins and vitamin derivatives, antihistamines, wetting agents, preservatives, moisturizers, emulsifiers, odorants, and the like, present in conventional concentrations. Those skilled in the art will have no difficulty in determining suitable materials and concentrations for their known functions. In addition, herbally derived agents can be included. Examples of herbal agents include feverfew, white willow bark, and goldenseal to name a few. In addition, analgesics and anti-inflammatories may also be included in the compositions and methods of the disclosure.

In one aspect a general formulation may comprise: NAD 0.2%; glutathione 0.2%; glycerin 25%; ascorbic acid (USP) 0.43%; potassium sorbate (USP) 0.15%, wherein the balance to 100% comprises water (e.g., about 74%) by weight. Where a saline solution is desirable the aqueous medium may comprise a small amount of dissolved sodium chloride in the aqueous medium. The salt concentration may be in the range of 0.1-2.0% and will typically be on the order of about 0.65% to 0.9%. The NaCl concentration may vary, but is typically at a normal physiological NaCl concentration. This general formulation may then be modified to include additional agents such as feverfew, white willow bark and the like, and/or an analgesic agent (e.g., an NSAID). The glycerin may be from any readily available source including natural sources as well as synthetic glycerin. In one aspect, the glycerin is a vegetable glycerin. The glycerin aids in the rapid uptake of the active agents and/or analgesics present in the compositions of the disclosure. For example, the glycerin may act by promoting vasodilation in the sublingual space thereby increasing the uptake of the herbal medicines and/or analgesics of the disclosure into the blood stream.

In one aspect, the disclosure provides methods and composition for buccal and sublingual administration. Sublingual administration offers advantages over other routes of administration. For example, compositions administered to the sublingual space have a rapid onset of action, reach high levels in the blood, avoid the first-pass effect of hepatic metabolism, and avoid exposure of the drug to fluids of the gastrointestinal tract. Additional advantages include easy access to the mucus membrane of the sublingual space so that an active substance contained in a therapeutic composition can be easily applied and localized. Further, there is good potential for prolonged delivery through the sublingual mucosal membrane (M. Rathbone & J. Hadgraft, 74 Int'l J. of Pharmaceutics 9 (1991)). Suitable nontoxic pharmaceutically acceptable carriers for use in the composition of the present buccal or sublingual dosages can be found in Remington's Pharmaceutical Sciences, 17th Edition, 1985. In addition, to the aqueous medium comprising the herbal medicine and/or analgesic, lozenges for buccal or sublingual administration may also be used. Formulations for lozenges are described in Modem Pharmaceutics, edited by G. S. Banker and C. T. Rhodes, 1996.

The sublingual mucosa includes the membrane of the ventral surface of the tongue and the floor of the mouth, whereas the buccal mucosa constitutes the lining of the cheek. Both the buccal mucosa and the sublingual mucosa are applicable to the methods and compositions of the disclosure, however, the sublingual space is typically targeted for delivery of the compositions of the disclosure. The sublingual mucosa is relatively more permeable than the buccal mucosa, thus giving rapid absorption and acceptable bioavailability of many active substances. Furthermore, the sublingual mucosa is convenient, accessible, and generally well accepted. This route has been investigated clinically for the delivery of a substantial number of drugs. It is a commonly used route for administration of nitroglycerin and is also used for buprenorphine and nifedipine (D. Harris & J. Robinson, 81 J. Pharmaceutical Sci. 1 (1992)).

The buccal mucosa is less permeable than the sublingual mucosa. The rapid absorption and high bioavailabilities seen with sublingual administration of drugs is not generally provided to the same extent by the buccal mucosa (D. Harris & J. Robinson, 81 J. Pharmaceutical Sci. (1992) at 2). The permeability of the oral mucosa is probably related to the physical characteristics of the tissues. The sublingual mucosa is thinner than the buccal mucosa, thus permeability is greater for the sublingual tissue. The palatal mucosa is intermediate in thickness, but is keratinized thus lessening its permeability, whereas the other two tissues are not.

The ability of molecules to permeate through the oral mucosa appears to be related to molecular size, lipid solubility, ionization and many other factors. Small molecules, less than about 100 daltons, appear to cross the mucosa rapidly. As molecular size increases permeability decreases rapidly. Lipid-soluble compounds are more permeable through the mucosa than are non-lipid-soluble molecules. In this regard, the relative permeability of molecules seems to be related to their partition coefficients. The degree of ionization of molecules, which is dependent on the pKₐ of the molecule and the pH at the membrane surface, also greatly affects permeability of the molecules. Maximum absorption occurs when molecules are un-ionized or neutral in electrical charge and absorption decreases as the degree of ionization increases. Therefore, charged drugs present a significant challenge to absorption through the oral mucosa.

Forms of delivery of compositions to the buccal mucosa and the sublingual mucosa include delivery by a lozenge, troche, breath freshener, mouthwash, or spray. These methods of delivery work by shedding or admixing the active ingredients in the composition into the saliva, which bathes the tissues of the oral cavity and throat as it passes posteriorly towards the esophagus. Such forms remain in the oral cavity only for short periods of time, generally not more than about 10 to 20 minutes.

Accordingly, in one aspect, the aqueous medium is designed for delivery to the buccal and/or sublingual mucosa. For example, delivery of the aqueous medium comprising an active/therapeutic agent of the disclosure such as, e.g. glutathione, NAD, and/or NADH, may be made by any conventional spray technique or device. Spray administration containers for various types of sublingual sprays are known and typically will be suitable for the disclosure for delivery of an aqueous composition comprising an active/therapeutic agent of the disclosure (e.g., glutathione, NAD, and/or NADH). The aqueous medium containing the composition of the disclosure will commonly be contained in a small bottle or similar container with a focused nozzle from which the aqueous medium comprising the herbal medicine and/or analgesic can be dispersed as a fine mist to be directed under the tongue. Using ambient air as the propelling agent, one can have the bottle made of a flexible plastic, so that merely squeezing the bottle's side propels the spray out through the nozzle into the sublingual space. Air is also the propelling agent for a pump sprayer, in which the user manipulates a small pump button which pumps air into the container and causes the liquid spray to be emitted on the return stroke. Alternatively, the bottle can be pressurized with a gas that is inert to the user and to the ingredients present in the aqueous medium. The gas will be dissolved under pressure in the container or may be generated by dissolution or reaction of a solid material that forms the gas as a product of dissolution or as a reaction product. Typical gases, which can be used, include nitrogen, argon, and a carbon dioxide.

The formulations described above and further herein can be used to treat diseases and disorders associated with oxidative damage. For example, the formulations can reduce the severity of oxidative damage and thus reduce symptoms associated with oxidative diseases and disorders.

Typically a subject will spray three to ten sprays of a formulation described herein at each administration, with the administration being repeated on an as needed basis. During use a subject need merely raise their tongue and direct a spray or drop comprising the formulation of the disclosure to the space under the tongue. The frequency of administration will be dependent on the nature of the usage. If administration is for relief of a current condition, such as a tremor, or trembling in hands, arms, legs, jaw, and face rigidity, or stiffness of limbs and trunk bradykinesia, or slowness of movement postural instability or impaired balance and coordination effects such relief may be expected within a few minutes of administration. Dosages may be repeated at intervals as the effect wears off or if the symptoms of tremor, or trembling in hands, arms, legs, jaw, and face rigidity, or stiffness of limbs and trunk bradykinesia, or slowness of movement postural instability or impaired balance and coordination persist. The user will normally discontinue administration once the symptoms subside. Administration can be resumed at a subsequent time when symptoms occur. In another aspect, the compositions of the disclosure may be administered at similar or smaller dosages and on a regular or less frequent basis to create a prophylactic effect intended to prevent the onset of a tremor, or trembling in hands, arms, legs, jaw, and face rigidity, or stiffness of limbs and trunk bradykinesia, or slowness of movement postural instability or impaired balance and coordination, or the like. Aqueous formulations, such as the above formulations, can be administered as drops, spray, aerosols or by any other dosage form. Optionally, the delivering system can be a unit dose delivery system. The volume of solution or suspension delivered per dose can be anywhere from 5 to 400 μl, typically between 50 to 150 μl. Delivery quantities of the composition for sublingual use are typically about 100 to 150 μl per spray.

In addition, to the treatment of Parkinson's disease and associated symptoms with NAD, NAD derivatives, and/or glutathione, the addition of herbal agents and/or analgesics in the compositions and methods of the disclosure can be used to treat aches and pains that may be associated with oxidative diseases and disorder. Examples of aches and/or pains that may be treated include headaches (e.g., migraines), toothaches, backaches, earaches and the like. Migraine headaches sometimes result in seizures due to suspected changes in blood flow. Similarly, the compositions and methods of the disclosure may prove useful in treating seizures associated with epilepsy by modulating blood flow.

Herbal medicines include those derived from, for example, feverfew, butterbur, dandelion, white willow bark, and the like. Feverfew (Tanacetum parthenium) is an herb in the Compositae family that has been known to have therapeutic properties (see, Bremness (ed.), “Herbs,” (1990), pp. 91:185-186 and Castleman, “The Healing Herbs,” (1991), pp. 173-176). Parthenolide is believed to be the active ingredient in feverfew and is thought to act by inhibiting the release of the vasoconstrictor serotonin from platelets. Recent studies suggest that parthenolide interacts with and inhibits IkappaB kinase beta (IKKbeta). This kinase subunit is known to play a critical role in cytokine-mediated signaling (see, e.g., Kwok et al., Chem Biol. 8(8):759-66, 2001). Parthenolide's in vitro and in vivo anti-inflammatory activity also appears to be mediated through the alpha-methylene gamma-lactone moiety shared by other sesquiterpene lactones. (Id.). Accordingly, feverfew may assist in migraine headache relief by inhibiting inflammation (e.g., via inhibiting release of inflammatory cytokines) and vasoconstriction/spasm thereby restoring normal blood flow.

Traditionally, feverfew has been administered as a raw leaf, either fresh or frozen, which is taken by chewing, by swallowing pills, tablets, capsules, by taking teas, or alcohol tinctures in which the feverfew is incorporated. It has also been administered as a tea with a concentration of 0.5-1 teaspoonfuls of feverfew per cup of boiling water. However, raw feverfew leaves are bitter and therefore unpleasant to chew and the tea is unpleasant to drink. Some evidence suggests that large amounts of feverfew cause oral ulcers or other irritations to the buccal membranes or mucosal membranes of the body including those of the mouth when taken at such high concentrations. In addition, the administration of feverfew by swallowing of the chewed material, drinking of tea, or swallowing of capsules, pills, or tinctures means that the feverfew must be released and dispersed to the central nervous system or other affected organs through the gastrointestinal system. Consequently, as discussed above, the active ingredients found in feverfew will not be readily available to a person to whom the herb has been administered. This has particularly significant drawback in the treatment of migraine headaches.

In the disclosure, feverfew is provided by either picking fresh leaves and allowing them to dry to a stage where they can be finely ground or otherwise comminuted, or by obtaining previously dried leaves, whole, or previously ground to a desired size. The dried plant (e.g., leaf) particles are dispersed or dissolved in an aqueous media such as the aqueous medium described herein below. The ground particle size useful in the compositions of the disclosure is about 0.1-20 μm, or 0.2-10 μm, but is typically about 0.2-5 μm. Alternatively, an extract of feverfew leaves may also be prepared by steam distillation, expression (hard pressing), or maceration. A tinture extract can be diluted as appropriate to obtain the desired concentration and/or therapeutic effect. Other methods of preparing the herbal extracts can be found in, “The Homoeopathic Pharmacopoeia,” Official Compendium, Jul. 1, 1992, Pharmacopoeia Convention of the American Institute of Homeopathy (Publishers), Falls Church, Va., incorporated herein by reference.

Incorporation of the feverfew particles or extract into the aqueous medium can be performed by dispersing or dissolving the feverfew as a 0.05 to 4% concentration in a lactone solution. When thoroughly mixed and dispersed and/or dissolved, the feverfew will be present at a concentration of about 0.001-2.0%, more commonly about 0.01-0.35%, but typically at about 0.10% by weight.

Similarly, the extract of the Butterbur plant (Petasites hybridus) has been used to treat headaches, neuralgia, and inflammation of the urethra. The extract has been taken orally as a tea or in tablet or gel cap form. The disclosure provides butterbur extract in an amount that is safe and efficacious for administration to the mucus membranes of a subject. The butterbur extract may be administered alone or in combination with the analgesics and/or herbal medicines (e.g., feverfew) as disclosed herein.

In the disclosure, butterbur extract is provided by utilizing the rhizomes, roots, and/or leaves of the butterbur plant. In one aspect, the roots and/or leaves are allowed to dry to a stage where they can be finely ground or otherwise comminuted, or by obtaining previously dried roots and/or leaves whole, or previously ground to a desired size. The dried plant (e.g., leaf) and/or root particles are dispersed or dissolved in an aqueous media such as the aqueous medium described herein below. The ground particle size useful in the compositions of the disclosure is about 0.1-20 μm, or 0.2-10 μm, but is typically about 0.2-5 μm. Alternatively an extract of the roots and/or leaves may also be prepared by steam distillation, expression (hard pressing), or maceration.

Incorporation of the butterbur particles or extract into the aqueous medium can be performed by dispersing or dissolving the butterbur as a 0.05 to 4% concentration in a lactone solution. When thoroughly mixed and dispersed and/or dissolved, the butterbur will be present at a concentration of about 0.001-2.0%, more commonly about 0.01-0.35%, but typically at about 0.10% by weight.

In addition, a number of natural analgesic and anti-inflammatory agents exist. White willow bark (a member of the sialix sp.), also known as natural aspirin, has been used in the treatment of pain, fever and as a topical antiseptic. An active ingredient in the white willow bark is salicin, which is converted by the body to acetylsalicylic acid, or aspirin. Although white willow bark is believed to act in a manner similar to aspirin by blocking prostaglandin synthesis, it is efficacious at a lower blood level than aspirin. Recent studies have reported a peak plasma level of 10 mM/L following administration of 1,360 mg extract containing 240 mg salicin. This plasma level is below that of 130 mM/L that occurs following the administration of 500 mg aspirin, a dose common for analgesic and antipyretic activity (see, Schmid et al. Eur J Clin Pharmacol. 57(5):387-91, 2001). In addition sodium salicylates may act by inhibiting the function of neutrophils, the most abundant cell associated with inflammation. Moreover, salicylates that lack an acetyl group, such as those present in white willow bark, do not inhibit aggregation of platelets at physiologically relevant concentrations (see, Krivoy et al., Planta Med. 67(3):209-12, 2001).

In the disclosure, white willow bark may be dried and ground in a manner similar to the feverfew leaves. The formulation is adjusted to contain 15% salicin. When thoroughly mixed and dispersed and or dissolved in the aqueous medium of the disclosure the white willow bark will commonly be present at a concentration of about 0.001-2.0% (e.g., about 0.05%), but is typically about 0.01-0.35% by weight.

In another aspect of the disclosure, nonsteroidal anti-inflammatory drugs (NSAIDs), such as, for example, aspirin, ibuprofen (Motrin, Advil, Rufen, others), naproxen, and the like; may be prepared and delivered in accordance with the methods and compositions of the disclosure. Such NSAIDS may be delivered either alone to the mucosal membranes or in combination with one or more other NSAIDs or herbal medicines and/or active ingredients thereof. The compositions and methods of delivery comprising the anti-inflammatory agents listed herein provide advantages including, for example, rapid uptake through the mucosal membrane of a subject as well as a need for smaller doses due to the route of administration.

The disclosure provides an aqueous medium comprising feverfew, white willow bark, butterbur, other herbal medicines, analgesic (e.g., NSAIDs), in combination with NAD, NAD derivatives, and/or glutathione.

The disclosure has described administration to the buccal and sublingual spaces, however, the formulations may be applied to the mucosal membranes of the airway. An airway is any part of the mammalian anatomy that air passes through during respiration including the mouth, nasal passages, trachea, bronchi, and bronchial tubes. Such airways are lined by mucosa and thus are applicable to the methods and compositions of the disclosure. The administration of an active/therapeutic agent of the disclosure to the lung airways can be any type of administration that places the therapeutic in contact with lung airway mucus membrane. Such administration can include, for example, by inhalations, nasal sprays, and nasal irrigations wherein the therapeutic contacts the lung airway mucus membrane. Typical devices for airway administration include a bulb, an inhaler, a nebulizer, an aerosol canister, a spray can, and a mask.

In another aspect of the disclosure, the aqueous medium is designed for delivery to the sinonasal cavity. In this aspect, an aqueous spray comprising the herbal medicine and/or analgesic is made to the nasal cavity by any conventional spray technique or device. Spray administration containers for various types of nasal sprays are known and are typically suitable for the delivery of the aqueous medium comprising the therapeutic of the disclosure. The aqueous liquid medium containing the glutathione, NAD, and/or NADH, and other appropriate ingredients will commonly be contained in a small bottle or similar container with a focused nozzle, from which it can be dispersed as a fine mist to be directed into each nostril. Using ambient air as the propelling agent, one can have the bottle made of a flexible plastic, so that merely squeezing the bottle's sides impels the spray out through the nozzle into the nasal cavity. Air is also the propelling agent for a pump sprayer, in which the user manipulates a small pump button which pumps air into the container and causes the liquid spray to be emitted on the return stroke. Alternatively, the bottle can be pressurized with a gas that is inert to the user and to the ingredients of the solution. The gas will be dissolved under pressure in the container or may be generated by dissolution or reaction of a solid material that forms the gas as a product of dissolution or as a reaction product. Typical gases, which can be used, include nitrogen, argon, and carbon dioxide.

Typically a subject will spray two or three sprays in each nostril at each administration, with the administration being repeated on an as needed basis. The frequency of administration will be dependent upon the nature of the usage. If administration is for relief of a current condition, such as tremor, or trembling in hands, arms, legs, jaw, and face rigidity, or stiffness of limbs and trunk bradykinesia, or slowness of movement postural instability or impaired balance and coordination, or the like, initial relief effects can be expected within a few minutes of administration. If such does not occur, a user may administer a second dosage. Dosages may be repeated at intervals as the effect wears off. A user will normally discontinue administration once the symptoms have subsided. Administration can be resumed at a subsequent time when another symptoms reoccur. The nasal spray can be administered regularly in smaller doses and on a less frequent basis, to create a prophylactic effect. Delivery quantities of the composition for sublingual use are typically about 100 to 150 μl per spray.

The nasal administration of the composition of the disclosure to the mucus membrane of a subject typically places the agent in contact with nasal-paranasal mucus. Direct administration to the nasal-paranasal anatomies can include, without limitation, nasal irrigations, nasal sprays, nasal inhalations, and nasal packs with, for example, saturated gauze provided that the administered composition contact the nasal-paranasal mucus prior to crossing the epithelium. Any device can be used to directly administer the herbal medicines and/or analgesics to the nasal-paranasal anatomy including, without limitation, a bulb, an inhaler, a canister, a spray can, a nebulizer, and a mask. For example, a 20 mL bulb can be used to irrigate the nasal-paranasal anatomy with an aqueous medium comprising a therapeutic agent or composition of the disclosure. Such an aqueous medium formulation can be stored at −20° C., 0° C., or room temperature. If stored below room temperature, the formulation typically is warmed prior to application to the nasal-paranasal cavities.

As noted above, the particular route of administration can influence the effective amount and duration of treatment with a therapeutic agent of the disclosure as well as the frequency of administration. For example, orally administered agents may require higher concentrations to deliver an effective amount to a target area or tissue than administration to a mucus membrane.

Other routes of administration to mucosal tissue of a subject include contacting mucosal tissue of the rectum, vagina, and eyes. For example, to treat abdominal cramping due to pre-menstrual syndrome (PMS) or other disease, disorder, or affliction, or to treat back pain, contacting the mucosal tissue most closely associated with the area may result in an increased relief of symptoms compared to oral analgesics and the like.

The compositions can be administered without invasive procedures (e.g., the use of needles for IV administration). Accordingly, subjects afflicted with Parkinson's disease can administer the therapeutic agents themselves without worrying about the dangers of needles. In addition, where a subject is so afflicted with Parkinson's or other disabling disease or disorder an assistant can easily administer drops or aerosols to the subject for relief of the various symptoms (e.g., Parkinson's symptoms including tremor, or trembling in hands, arms, legs, jaw, and face rigidity, or stiffness of limbs and trunk bradykinesia, or slowness of movement postural instability or impaired balance and coordination).

A number of embodiments have been described. Nevertheless, it will be understood that various modifications may be made without departing from the spirit and scope of the disclosure. Accordingly, other embodiments are within the scope of the following claims.

What is claimed is:

1. A composition comprising an aqueous medium having dispersed or dissolved therein
a glutathione and an NAD or derivative thereof, wherein the glutathione is at an amount from about 0.01% to 0.9%, and wherein the NAD or derivative thereof is at an amount from about 0.01% to 0.9%.

2. A composition of claim 1, wherein the composition further comprises an analgesic selected from the group consisting white willow bark, aspirin, ibuprofen, naproxen, and any combination thereof in an amount of from about 0.001% to about 0.35% by weight; and
feverfew dried plant particles dispersed or dissolved in the aqueous medium having a concentration of about 0.001 to 2.0% in the aqueous medium.

3. The composition of claim 1, wherein the NAD or derivative thereof is nicotinamide adenine dinucleotide (NAD); nicotinamide adenine dinucleotide phosphate (NADP); or nicotinamide adenine dinucleotide, reduced form (NADH).

4. The composition of claim 1, wherein the NAD or NAD derivative is selected from the group consisting of quinolinic acid; quinolinic acid ribonucleotide; nicotinamide; nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

5. The composition of claim 1, wherein the glutathione is tripeptide L-glutathione (GSH) (gamma-glutamyl-cysteinyl-glycine or a dimer (GSSG).

6. The composition of claim 1, wherein the glutathione amount is 0.2%.

7. The composition of claim 2, wherein the feverfew is present at a concentration of about 0.01% to 0.35%.

8. The composition of claim 7, wherein the feverfew is present at a concentration of 0.10%.

9. The composition of claim 1, wherein the composition further comprises an agent selected from the group consisting of butterbur extract, goldenseal extract, dandelion extract, polyporous extract, ascorbic acid, potassium sorbate, and any combination thereof.

10. The composition of claim 1, further comprising a material selected from the group consisting of a surfactant, a vitamin, a vitamin derivative, a wetting agent, a preservative, and an emulsifier.

11. The composition of claim 10, wherein the emulsifier is glycerin.

12. The composition of claim 2, wherein the analgesic agent is white willow bark.

13. A method of treating a subject suffering from an oxidative disease or disorder, comprising administering a composition of claim 1.

14. A method of treating as subject suffering from Parkinson's disease, comprising administering to a mucus membrane of the subject a composition comprising: an aqueous medium; a glutathione and an NAD or derivative thereof, wherein the glutathione is at an amount from about 0.01% to 0.9%, and wherein the NAD or derivative thereof is at an amount from about 0.01% to 0.9% in the aqueous medium; and wherein the composition is administered in an amount effective to reduce the severity and/or treat the symptoms of Parkinson's disease.

15. The method of claim 14, wherein the composition further comprises an analgesic selected from the group consisting white willow bark, aspirin, ibuprofen, naproxen, and any combination thereof in an amount of from about 0.001% to about 0.35% by weight; and
feverfew dried plant particles dispersed or dissolved in the aqueous medium having a concentration of about 0.001 to 2.0% in the aqueous medium.

16. The method of claim 14, wherein the NAD or derivative thereof is nicotinamide adenine dinucleotide (NAD); nicotinamide adenine dinucleotide phosphate (NADP); or nicotinamide adenine dinucleotide, reduced form (NADH).

17. The method of claim 14, wherein the NAD or NAD derivative is selected from the group consisting of quinolinic acid; quinolinic acid ribonucleotide; nicotinamide; nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

18. The method of claim 14, wherein the glutathione is tripeptide L-glutathione (GSH) (gamma-glutamyl-cysteinyl-glycine or a dimer (GSSG).

19. The method of claim 14, wherein the glutathione amount is 0.2%.

20. The method of claim 15, wherein the feverfew is present at a concentration of about 0.01% to 0.35%.

21. The method of claim 20, wherein the feverfew is present at a concentration of 0.10%.

22. The method of claim 14, wherein the composition further comprises an agent selected from the group consisting of butterbur extract, goldenseal extract, dandelion extract, polyporous extract, ascorbic acid, potassium sorbate, and any combination thereof.

23. The method of claim 14, further comprising a material selected from the group consisting of a surfactant, a vitamin, a vitamin derivative, a wetting agent, a preservative, and an emulsifier.

24. The method of claim 23, wherein the emulsifier is glycerin.

25. The method of claim 15, wherein the analgesic agent is white willow bark.

26. A nasal spray comprising the composition of claim 1.

27. A sublingual spray comprising the composition of claim 1.

28. The composition of claim 1, wherein the NAD or NAD derivative is at about 0.2%.

29. The method of claim 14, wherein the NAD or NAD derivative is at about 0.2%.
